SEQUENCE LISTING (2 sequences)

SEQ ID NO: 1 - RNA, 43 nt, Artificial Sequence
  Synthetic inhibitory nucleic acid sequence
gcacugaagucagccuuauuurauaaggcugacuucagugcag

SEQ ID NO: 2 - RNA, 43 nt, Artificial Sequence
  Synthetic inhibitory nucleic acid sequence
cggugauuuugggccuauuuuraauaggcccaaaaucaccguc

(19) United States
(12) Patent Application Publication
(10) Pub. No.: US 2010/0008900 A1
(43) Pub. Date: Jan. 14, 2010

(54) ANNEXIN II COMPOSITIONS FOR TREATING OR MONITORING INFLAMMATION OR IMMUNE-MEDIATED DISORDERS

(75) Inventors: Daniel Tak Chan, Hong Kong (HK); Susan Sou Ying Yung, Pokfulam (HK)

Correspondence Address: Pabst Patent Group LLP, 1545 PEACHTREE STREET NE, SUITE 320, ATLANTA, GA, 30309, US

(73) Assignee: The University of Hong Kong

(21) Appl. No.: 12/172,665

(22) Filed: Jul. 14, 2008

(51) Int. Cl. A61K 39/395; A61K 38/02; A61K 31/711; C12Q 1/02; A61P 37/00; A61P 29/00

(52) U.S. Cl. 424/130.1; 514/2; 435/29; 514/44 A

(57) ABSTRACT

Methods and compositions for treating, detecting, diagnosing or assisting in the diagnosis of an inflammatory or immunological disorder are provided. The authors have made the novel discovery that anti-dsDNA antibodies are reactive to cell surface annexin II and through this interaction, anti-dsDNA antibodies are bound and then internalized and translocated into the cytoplasm and/or nucleus where they mediate altered cellular functions. Compositions that inhibit or interfere with the interaction of annexin II with anti-dsDNA antibodies can be used to treat one or more symptoms or pathological processes of an inflammatory or immunological disorder, preferably an auto-immune disorder. Methods for monitoring the progression or severity of an inflammatory or immunological disorder are also provided. In certain embodiments, levels of annexin II and/or anti-dsDNA antibodies, including anti-dsDNA antibodies that can bind to annexin II, are assessed and compared to a reference level of annexin II and/or anti-dsDNA antibodies that corresponds to a specific stage or severity of the disorder.

### FIELD OF THE INVENTION

The invention is generally related to methods and compositions that assist in the diagnosis of an immune disorder as well as methods and compositions for the treatment of one or more symptoms of an immune disorder.

### BACKGROUND OF THE INVENTION

Autoimmunity occurs when an individual's immune system turns on itself and targets its own organs, tissues and cells for destruction. Systemic Lupus Erythematosus (“SLE” or “Lupus”) is a prototype, multi-system autoimmune disease characterized by the production of numerous auto-antibodies, and can affect the skin, kidneys, lungs, central nervous system and blood vessels resulting in arthritis, serositis, and renal, neurologic and blood disorders. Pathogenesis commences with impaired immune regulation and abnormal B and T cell activation and the generation of auto-antibodies. Auto-antibodies are immunoglobulins that bind to “self” structures. Thereafter, infiltration by T and B cells, deposition of auto-antibodies, in particular anti-dsDNA antibodies, and the formation of immune complexes in tissues trigger local inflammatory processes, fibrosis and ultimately organ damage.

Kidney involvement (lupus nephritis) occurs in over half of the SLE population and contributes significantly to patient morbidity and mortality, leading to acute or chronic renal failure. Auto-antibodies of the IgG class that show binding activity to double-stranded (ds) DNA are a hallmark of SLE, in particular in those patients who develop lupus nephritis. Active disease often correlates with elevated levels of circulating anti-dsDNA antibodies, and the presence of these auto-antibodies can pre-date disease manifestations by many years.

The importance of intra-renal deposition of anti-dsDNA antibodies in the pathogenesis of lupus nephritis is highlighted by the observation that they can be eluted from renal specimens obtained from both lupus patients and lupus mice. Furthermore, many features of lupus nephritis can be replicated in non-autoimmune mice after intra-peritoneal administration of human or murine anti-dsDNA antibodies, or inoculation with the transgene that encodes the secreted form of an IgG anti-dsDNA antibody.

The onset of lupus nephritis follows the deposition of anti-dsDNA antibodies in the kidney parenchyma, especially the glomeruli, and possibly the tubulo-interstitial areas. Data to date suggest two possible binding mechanisms—that they may bind either indirectly to involved tissue through chromatin material (DNA, nucleosomes, histones) or directly to cell surface antigens. Recent data suggests that anti-dsDNA antibodies may “cross-react” with various antigens on the cell surface and in the extracellular matrix. See, for example, U.S. Pat. No. 5,681,700 and U.S. Pat. No. 6,342,218 to Reichlin, et al., which describe anti-dsDNA antibodies cross-reactive with A and D SnRNP proteins and anti-dsDNA antibodies which are cross-reactive with ribosomal protein S1.

Although the treatment of lupus nephritis has improved significantly over the past decade, current treatment regimens remain in the realm of non-specific immunosuppression, due to inadequate knowledge of detailed pathogenic processes. Several side-effects are associated with non-specific immunosuppression, including increased susceptibility to infection and specific toxicities of individual medications. In addition to immunosuppressive actions, prednisone treatment is associated with acne, striae, cushingoid facies, truncal obesity, easy bruising, hyperglycemia, hyperlipidemia, hypertension, osteoporosis, and weight gain. Cyclophosphamide causes marrow suppression, gonadal toxicity, alopecia, hemorrhagic cystitis, and is associated with an increased risk of cancer. Mycophenolate mofetil causes anemia, diarrhea and vomiting. Cyclosporine and tacrolimus cause hypertension, nephrotoxicity, gingival hyperplasia, hirsutism, hyperlipidemia, and hyperglycemia.

Therefore, it is an object of the invention to provide methods and compositions for treating one or more symptoms of an immunological disorder.

It is another object of the invention to provide methods and compositions that assist in the diagnosis of an immunological disorder.

### SUMMARY OF THE INVENTION

Methods and compositions are provided that assist in the diagnosis of immunological disorders or treatment of one or more symptoms of an immunological disorder, preferably an autoimmune disorder or an inflammatory disorder, in particular an inflammatory disorder involving anti-dsDNA antibodies, including but not limited to those which are internalized by cells and thereby cause changes in cell function or cell damage. One embodiment provides methods and compositions that assist in the diagnosis or the treatment of Systemic Lupus Erythematosus (SLE or Lupus). The authors have discovered that anti-dsDNA antibodies bind to cell surface annexin II. The interaction between anti-dsDNA antibodies and annexin II can be associated with internalization of anti-dsDNA antibodies and their translocation to the cells' cytoplasm or nuclei with subsequent changes in cell function, including the induction of inflammatory mediators such as interleukin-6 (IL-6). Exemplary cells that internalize anti-dsDNA antibodies include cells that express annexin II on the outer surface of the cell membrane such as kidney mesangial cells.

Another embodiment provides compositions that inhibit or reduce the internalization of anti-dsDNA antibodies by inhibiting or reducing the binding of anti-dsDNA antibodies to annexin II. Inhibition of the interaction between anti-dsDNA antibodies and annexin II can be accomplished using compounds that bind to either the anti-dsDNA molecules or annexin II. Such molecules can be polypeptides such as antibodies to annexin II, other molecules that interact with annexin II, or soluble forms of annexin II. Alternatively, expression of annexin II can be down-regulated. By down-regulating the available amount of annexin II, it is believed that the cellular binding by anti-dsDNA antibodies and the internalization of anti-dsDNA antibodies via annexin II will be decreased. Downregulation of annexin II can be accomplished using inhibitory nucleic acids including dsRNA, siRNA, microRNA, antisense oligonucleotides, and triplex forming oligonucleotides that bind to mRNA or DNA encoding annexin II.

Another embodiment provides a method for treating one or more symptoms of an immunological disorder by administering to a subject an effective amount of an annexin II ligand to inhibit cellular internalization of anti-dsDNA antibodies by the subject. In a preferred embodiment, the immunological disorder is SLE or lupus nephritis. An exemplary annexin II ligand is an antibody specific to annexin II. The antibody is preferably a single chain or humanized antibody or antigen binding fragment thereof. Another example of such a ligand is a non-peptide molecule that interferes with the interaction between annexin II and anti-dsDNA antibodies.

Still another embodiment provides a method for diagnosing or assisting in the diagnosis of an immunological disorder in a subject by assaying a sample from the subject for anti-dsDNA antibodies that demonstrate the ability to bind to annexin II. An increase in levels of anti-dsDNA antibodies that bind to annexin II compared to a control is indicative of an immunological disorder or can assist in the diagnosis of an immunological disorder when considered in conjunction with one or more other symptoms of an immunological disorder.

Another embodiment provides a method for diagnosing or assisting in the diagnosis of an immunological disorder in a subject by assaying the amount of annexin II in a sample from the subject. An increase in the amount of annexin II or soluble annexin II relative to a control is indicative of or assists in the diagnosis of an immunological disorder.

Still another embodiment provides a method for determining the progression or stage of an immunological disorder by determining the levels of anti-dsDNA antibody that binds DNA and annexin II in a subject and comparing the levels or the anti-dsDNA antibody with a control wherein the level of anti-dsDNA antibody in the subject is indicative of the progression or stage of the immunological disorder. Alternatively, the levels of annexin II can be used to determine the progression or stage of the immunological disorder. In certain embodiments, the levels of anti-dsDNA antibodies that bind to annexin II and the levels of annexin II in a host can both be used to determine the progression or stage of the immunological disorder.

### BRIEF DESCRIPTION OF THE DRAWINGS

FIG. 1 shows a mass spectroscopy spectrum of the 36 kDa cell protein to which anti-dsDNA antibodies bind, which was identified as annexin II.

FIG. 2A is a graph showing binding of immunoglobulins of the IgG class to annexin II (OD) in control serum (▪), serum from patients with inactive lupus nephritis (◯), or serum from patients with active lupus nephritis (). FIG. 2B is a graph showing binding of serum IgG to annexin II (OD) and their association with anti-dsDNA Ab levels (IU/ml) in serum from patients with inactive lupus nephritis (◯) or serum from patients with active lupus nephritis ().

FIG. 3A is a graph showing binding to annexin II (OD) of control human Ig (▪), isolated anti-dsDNA antibodies from patients with inactive lupus nephritis (◯), or anti-dsDNA antibodies from patients with active lupus nephritis (). FIG. 3B is a graph depicting anti-dsDNA antibody binding to annexin II (OD) and their correlation with anti-dsDNA Ab levels (IU/ml) for anti-dsDNA antibody preparations from patients with inactive lupus nephritis (◯) or anti-dsDNA Ab levels from patients with active lupus nephritis ().

FIG. 4 is a graph depicting the concentration of IL-6 (ng/μg cellular protein) secreted by human mesangial cells stimulated with serum free medium (▴), control human IgG (◯), and anti-dsDNA antibodies ().

FIG. 5 is a graph showing the level of IL-6 (ng/μg cellular protein) secreted by human mesangial cells which have or have not been treated with annexin II RNAi and then stimulated with anti-dsDNA Ab from patients with inactive lupus nephritis (◯), or anti-dsDNA Ab from patients with active lupus nephritis ().

### DETAILED DESCRIPTION OF THE INVENTION

### I. Definitions

To assist in a clear understanding of the specification and claims, the following definitions are provided:

The term “protein” refers to polymers of amino acids which may, or may not, include all post-translational modifications such as processing, dimerization, truncations, substitutions, deletions or insertion in the amino acid sequence, glycosylations and phosphorylations which often play crucial roles in modulating the function of the protein. The term “protein” includes natural proteins, synthetic or recombinant proteins, polypeptides or peptides.

The term “protein modification” or “post-translational modification” includes qualitative changes in the protein annexin II. Such modifications can include, but are not limited to, changes in the amino acid sequence, transcriptional or translational splice variation, pre- or post-translational modifications to the DNA or RNA sequence, addition of macromolecules or components to the DNA, RNA or protein such as peptides, ions, sugar-containing molecules, and lipid-containing molecules.

The term “antibody” as used herein refers to the intact molecule of immunoglobulin as well as the antigen binding fragments thereof, and includes Fab, Fab′, and F(ab)₂, which are capable of binding the epitopic determinant. The Fab fragment contains a monovalent antigen-binding fragment of an antibody molecule, and can be generated by the enzymatic digestion of the whole antibody with papain to yield an intact light chain, and a portion of one heavy chain. The Fab′ fragment of an antibody is generated by treatment of the whole antibody with the enzyme pepsin, followed by reduction, to yield an intact light chain and a portion of the heavy chain. This method of digestion yields two Fab′ fragments per one antibody molecule. The F(ab′)₂ fragment of the antibody is generated by enzymatic treatment of the whole antibody with pepsin without subsequent reduction. F(ab)₂ is a dimer of two Fab′ fragments which are held together by two disulfide bonds.

The term “auto-antibody” refers to an immunoglobulin that is directed against one or more of the individual's own (i.e., self) proteins.

The term “autoimmune disease” refers to the failure of an individual to recognize its own constituent parts as self, and which results in an immune response against the individual's own cells and tissues, causing damage and injury to tissues and organs.

The terms “immune-mediated diseases” or “immunological disorder” are used interchangeably and refer to diseases in which immunoglobulins or lymphocytes play a role in causing one or more symptoms and the tissue pathology.

The term “inflammation” refers to the body's reaction to injury or infection and is characterized by the infiltration of the body's white blood cells to sites of injury, swelling, and generation of inflammatory mediators to facilitate healing.

The term ‘inflammatory diseases’ refers to all diseases characterized by acute or chronic inflammation, with the increased synthesis of the pro-inflammatory mediators such as IL-6 and others, and can include, but are not limited to, diseases such as immune-mediated inflammatory/fibrotic diseases (SLE, rheumatoid arthritis, kidney transplantation, Crohn's disease, Castleman's disease) that affect the kidney, liver, gut, joints, or are non-specific; respiratory diseases with prominent inflammation and fibrosis that include bronchial asthma, and chronic obstructive pulmonary disease); systemic diseases with inflammation and fibrosis playing a significant role in organ injury and includes blood vessels in atherosclerosis, blood vessels and the kidney in hypertension, blood vessels and the kidney in diabetes mellitus; and inflammatory and fibrotic responses in cancer.

The term “enzyme-linked immunosorbent assay” or “ELISA” is a biochemical technique used predominantly in the field of immunology to detect the presence of an antibody or an antigen in a biological sample.

The term “immunoassay” refers to a biochemical test that measures the concentration of a substance in a biological sample, and utilizes the reaction of an antibody or antibodies to its antigen. Immunoassays may be used to detect the presence of either the antigen or antibody.

The term “biological sample” refers to a fluid or tissue sample from a subject. Fluid samples include, but are not limited to saliva, tears, urine, whole blood, plasma, lymphatic fluid, interstitial fluid, and cerebrospinal fluid.

The term “effective amount” or “therapeutically effective amount” means a dosage sufficient to provide treatment of the inflammatory response, immunological or autoimmune disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

II. Compositions for Inhibiting the Binding of Anti-dsDNA Antibodies to Cells or the Internalization of Anti-dsDNA Antibodies into Cells.

Compositions and methods for inhibiting or reducing the binding by or internalization of anti-dsDNA antibodies into cells relative to a control not including the composition are provided. It is believed that reducing the binding by or internalization of anti-dsDNA antibodies can be an effective method in treating some anti-inflammatory disorders and immunological disorders, particularly autoimmune disorders. Preferred compositions are those that inhibit or reduce the binding of anti-dsDNA antibodies to annexin II, preferably to annexin II on the surface of a cell. These inhibitory compositions can be ligands of annexin II that compete with anti-dsDNA antibodies for binding to annexin II on cell surfaces. Alternatively, these compositions can bind to anti-dsDNA antibodies and prevent the anti-dsDNA antibodies from binding to annexin II on cell surfaces. Small molecules can also be used to interfere with the interaction of anti-dsDNA antibodies with annexin II on cell surfaces. Still another embodiment provides inhibitory nucleic acids that can down regulate the expression of annexin II in a subject and thereby inhibit or prevent the cellular binding by anti-dsDNA antibodies or internalization of anti-dsDNA antibodies into the cells of the subject.

A. Ligands of Annexin II or Anti-dsDNA Antibodies

The annexins constitute a family of calcium-dependent phospholipid-binding proteins widely expressed in the animal and plant kingdom. In humans, twelve annexin proteins have been identified to date and their expression is tissue- and cell-type specific. Members of the annexin family are composed of a highly conserved planar array of four or eight homologous domains that contain 70-amino acid long repeats, each containing five α-helices. The annexin proteins have been implicated in various physiological functions that include the trafficking of membrane proteins, regulation of membrane bound ion channels, and possess anti-inflammatory, fibrinolytic and anti-coagulatory properties. Increased annexin expression has been associated with various diseases, which include lung cancer, multiple sclerosis and cardiovascular diseases. These diseases have been attributed to post-translational or protein modifications.

1. Antibodies

Suitable antibodies may be recombinant, humanized or non-humanized, polyclonal or monoclonal. These may be antigen binding fragments, single chain, dimeric or multimeric. Methods for producing antibodies are well known in the art. See Antibodies: A Laboratory Manual, Ed Harlow and David Lane eds., (1988), Cold Spring Harbor Laboratory Press, Cold Spring Harbor, N.Y.

a. Annexin II

A preferred embodiment provides compositions including an effective amount of antibody or antigen binding fragment thereof that binds to annexin II on cell surfaces and interferes, inhibits, or reduces the binding and/or internalization of anti-dsDNA antibodies into cells expressing annexin II on their surface. The antibody or annexin II binding fragment thereof effectively competes with circulating anti-dsDNA antibodies for binding sites on annexin II.

The amino acid and cDNA sequence for human annexin is known in the art, see for example NCIB Accession Number AAH66955 and B1C066955. There are commercially available antibodies with specificity for annexin II.

b. Anti-dsDNA Antibodies

Another embodiment provides a composition having an effective amount of an antibody or antigen binding fragment thereof to bind to anti-dsDNA antibodies circulating in a subject. The binding of antibodies to the anti-dsDNA antibodies in the subject's circulatory system interferes with or inhibits the association of the anti-dsDNA antibodies to annexin II on cell surfaces, regardless of the region of immunoglobulin that these antibodies interact with.

2. Small Molecules

Compositions containing small molecular ligands of annexin II or anti-dsDNA antibodies are also provided. The term “small molecule” refers to molecules having a molecular weight below about 500 Daltons. Typically small molecule ligands of annexin II are obtained by screening a library of compounds for binding ability to annexin II. Representative small molecule ligands are those having at least one carbon ring structure.

3. Fusion Proteins

Fusion proteins having a first polypeptide operably linked to second polypeptide are also provided. An exemplary fusion protein includes part or all of annexin II protein fused (i) directly to a second polypeptide or, (ii) optionally, fused to a linker peptide sequence that is fused to the second polypeptide.

The first polypeptide typically includes a fragment of annexin II that is bound by anti-dsDNA antibodies. Anti-dsDNA antibodies can be obtained using DNA affinity chromatography as described in Kubota, T. et al. Clin. Exp. Immunol., 62(2): 321-328 (1985). The isolated anti-dsDNA antibodies can then, be screened for the ability to bind to annexin II using conventional techniques. Once the anti-dsDNA antibodies that cross-react with annexin II are isolated, the antibodies can be screened with various fragments of annexin II to isolate fragments of annexin II that bind to the anti-dsDNA antibodies. The fragments can be sequenced and that sequence can be incorporated into a fusion protein expression vector.

A second polypeptide, preferably an immunoglobin fragment is operably linked to the first polypeptide such that each polypeptide retains its biological function. In certain embodiments the second polypeptide is a heterologous polypeptide, i.e., obtained from a different host or species than the first polypeptide. In one embodiment, the second polypeptide includes one or more domains of an Ig heavy chain constant region, preferably having an amino acid sequence corresponding to the hinge, C_{H2} and C_{H3} regions of a human immunoglobulin Cγ1 chain.

4. Soluble Annexin II Fragments

In another embodiment, the compositions include an effective amount of a soluble annexin II fragment that is capable of binding to anti-dsDNA antibodies and preventing or inhibiting the anti-dsDNA antibody from binding annexin II on cell surfaces. The soluble annexin II fragment can be obtained as described above. It is believed that administering soluble annexin II fragments that can bind anti-dsDNA antibodies will reduce the binding of anti-dsDNA antibodies to cells and the internalization of anti-dsDNA antibodies, by interfering with the interaction of anti-dsDNA antibodies and annexin II on cell surfaces.

B. Inhibitory Nucleic Acids

In still another embodiment, the compositions contain an inhibitory nucleic acid in an amount effective to downregulate expression of annexin II. Exemplary inhibitory compositions include polypeptides, antibodies and antigen binding fragments thereof; inhibitory nucleic acid including dsRNA, siRNA, microRNA or antisense DNA that binds nucleic acids encoding annexin II. While the optimum length of the dsRNA may vary according to the target gene and experimental conditions, the duplex region of the RNA may be at least 19, 20, 21-23, 25, 50, 100, 200, 300, 400 or more bases long. Exemplary inhibitory nucleic acid sequences include, but are not limited to:

(SEQ ID NO:1)

r(GCACUGAAGUCAGCCUUAUUU)/r(AUAAGGCUGACUUCAGUGCUG)
and

(SEQ ID NO:2)

r(CGGUGAUUUUGGGCCUAUUUU)/r(AAUAGGCCCAAAAUCACCGUC)

from Qiagen (Ma G et al, J exp Med; 200: 1337-1346 (2004)).

C. Pharmaceutical Compositions

Pharmaceutical compositions including one or more ligands of annexin II or anti-dsDNA antibodies, and vectors encoding the same are provided. The pharmaceutical compositions may be for administration by oral, parenteral (intramuscular, intraperitoneal, intravenous (IV) or subcutaneous injection), transdermal (either passively or using iontophoresis or electroporation), transmucosal (nasal, pulmonary, vaginal, rectal, or sublingual) routes of administration or using bioerodible inserts and can be formulated in dosage forms appropriate for each route of administration.

1. Formulations for Parenteral Administration

In a preferred embodiment, the ligands are administered in an aqueous solution, by parenteral injection. The formulation may also be in the form of a suspension or emulsion. In general, pharmaceutical compositions are provided including effective amounts of a ligand for annexin II or anti-dsDNA antibodies, or derivative products, and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.
2. Formulations for Enteral Administration

Ligands for annexin II or anti-dsDNA antibodies can be formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The ligands may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience; New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The ligand for annexin II or anti-dsDNA antibodies can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e. the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), EudragitL30D, Aquateric®, cellulose acetate phthalate (CAP), Eudragit® L, Eudragit® S, and Shellac. These coatings may be used as mixed films.

3. Topical Delivery Formulations

The disclosed compositions can be applied topically. This does not work well for most peptide formulations, although it can be effective especially if applied to the lungs, nasal, oral (sublingual, buccal), vaginal, or rectal mucosa. The ligand for annexin II or anti-dsDNA antibody can be delivered to the lungs while inhaling and traverses across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent™ nebulizer (Mallinckrodt Inc., St. Louis, Mo.); the Acorn II™ nebulizer (Marquest Medical Products, Englewood, Colo.); the Ventolin™ metered dose inhaler (Glaxo Inc., Research Triangle Park, N.C.); and the Spinhaler™ powder inhaler (Fisons Corp., Bedford, Mass.).

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers.

4. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of ligands to annexin II or anti-dsDNA antibody, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release 5,13-22 (1987); Mathiowitz, et al., Reactive Polymers 6, 275-283 (1987); and Mathiowitz, et al., J. Appl. Polymer Sci. 35, 755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### III. Methods of Diagnosis and Treatment

Compositions and methods for assisting in the diagnosis of an inflammatory response or an immunological disorder are also provided. It has been discovered that annexin II protein expression is increased in subjects having an inflammatory or immunological disorder. For example, the kidneys of patients and animals with lupus nephritis, in particular the mesangial cells, have increased expression of annexin II relative to subjects without lupus nephritis. Thus, one embodiment provides a method for diagnosing an inflammatory disorder or an immunological disorder by obtaining a sample from a subject and determining the levels of annexin II in the sample, wherein elevated levels of annexin II relative to a control are indicative of an inflammatory or immunological disorder. An exemplary control is a sample from a subject that does not have an inflammatory or immunological disorder.

Still another embodiment provides a method for determining the progression or severity of an inflammatory or immunological disorder by determining the levels of anti-dsDNA antibodies that exhibit the ability to bind with annexin II, determining the levels of annexin II in a subject, and determining the levels of immunoglobins that bind to annexin II in the subject and comparing these levels to levels of a control that does not have inflammation or an immunological disorder (i.e., a negative control) or one with a known inflammatory or immunological disorder (i.e., a positive control). The levels of anti-dsDNA antibodies that bind with annexin II can also be used to assess the efficacy of treatments for inflammatory or immunological disorders. Levels of annexin II and immunoglobins that bind to annexin II in subjects known to have an inflammatory or immunological disorder can be used as indicators of the inflammatory or immunological disorder.

Still another embodiment provides a method for monitoring the progression of an inflammatory or immunological disorder by monitoring the levels of annexin II, immunoglobins that bind to annexin II, and anti-dsDNA antibodies over time. Samples can be taken from a subject periodically over months or years. If the subject is also being treated for an inflammatory or immunological disorder, the data can be used to evaluate treatment regimens designed to improve patient welfare. For example, if the expression of annexin II decreases over time during treatment, the treatment regime would be considered effective. If the levels of annexins were unchanged or increased, the treatment would be considered ineffective.

Representative inflammatory responses or immunological diseases or disorders that can be detected, assessed for severity, or treated include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, CREST syndrome, Crohn's disease, Dego's disease, dermatomyositis, dermatomyositis-juvenile, discoid lupus, essential mixed cryoglobulinemia, fibromyalgia-fibromyositis, Grave's disease, Guillain-Barre, Hashimoto's thyroiditis, idiopathic pulmonary fibrosis, idiopathic thrombocytopenia purpura (ITP), IgA nephropathy, insulin dependent diabetes (Type I), juvenile arthritis, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

A. Methods for Detecting or Quantifying Annexin II Levels

The detection of annexin II proteins in a biological sample obtained from a given subject is made possible by a number of conventional methods. A preferred method includes immunoassays whereby annexin II proteins are detected by their interaction with an annexin II specific antibody. Annexin II specific antibodies can be used to detect the presence of annexin II protein or its fragments in either a qualitative or quantitative manner. In addition, compounds other than antibodies, such as molecules that specially bind to annexin II protein can also be used to detect and assess the level of annexin II protein. These proteins include, but are not limited to, S100A proteins, plasminogen, and phospholipase A. Exemplary immunoassays that can be used for the detection of annexin II protein include, but are not limited to, radioimmunoassay, ELISAs, immunoprecipitation assays, Western blot, and fluorescent immunoassays.

A biological sample that may contain annexin II protein can be obtained with informed consent from an individual suspected or confirmed of having an autoimmune disease or inflammatory disease. If the biological sample is of tissue or cellular origin, the sample is solubilized in a lysis buffer optionally containing a chaotropic agent, detergent, reducing agent, buffer, and salts.

Immuoassays for the detection of annexin II protein include the ability to contact the biological samples with an antibody specific to annexin II protein under conditions such that an immunospecific antigen-antibody interaction may occur, followed by the detection or measurement of this interaction. The binding of the antibody to annexin II protein may be used to detect the presence and altered production of annexin II whereby the detection of altered levels of annexin II protein in a biological sample may be indicative of disease. The levels of annexin II protein is compared to standards obtained from healthy subjects, and from subjects without autoimmune or inflammatory diseases.

The detection of annexin II protein levels in biological samples can be useful in monitoring the efficacy of drugs or compounds or procedures used in the treatment of patients with immune-mediated or inflammatory diseases, as indicated by their actions on the level of annexin II. One embodiment provides an assay or kit that includes reagents for the detection and qualitative or quantitative measurement of annexin II in a subject's biological sample. For example, if detection of annexin II is by means of ELISA, components of the assay or kit will include an antibody directed against specific epitopes of the annexin II, in which the antibody may optionally be linked to an enzyme, fluorescent dye or radioactive label.

B. Methods to Measure Immunoglobulin Binding to Annexin II

Diagnostic and prognostic methods for autoimmune or inflammatory diseases are also provided. The diagnostic and prognostic methods include detecting antibodies to annexin II which optionally have cross-reactivity with DNA. An immunoassay or immuno-affinity chromatography can be performed by a method that allows the contact of a biological sample obtained from a subject with a separate sample that contains annexin II, under conditions such that an immunospecific antigen-antibody interaction occurs. The immunoassay or immunoaffinity chromatography protocol can be conducted through a number of methods, One method involves anchoring annexin II to a stationary solid support, passage of the biological sample which contains antibodies to annexin II through the solid phase, and detecting antibodies to annexin II that are bound to the solid support. Annexin II for anchoring to the stationary solid support can be prepared using recombinant DNA technology in which a DNA molecule encoding a fragment of annexin II can be genetically engineered into an appropriate expression vector. Alternatively, annexin II can be isolated and purified from natural sources using methodologies that are well known in the art and include but are not limited to gel filtration chromatography, ion-exchange chromatography, and gel electrophoresis. The antibody can then be eluted from the solid support for example by changing the concentration of salts in the elution buffer.

The detection of annexin II-binding immunoglobulins (antibodies) can be achieved using a second antibody that is specific for immunoglobins, for example, reactive with the Fc region of immunoglobins. The second antibody can be linked or conjugated to an enzyme. The enzyme which is bound to the antibody can react with an appropriate substrate such as a chromogenic substrate to produce a chemical reaction that can be detected, for example, by spectrophotometry. Enzymes that are commonly used to label antibodies include, but are not limited to, horseradish peroxidase and alkaline phosphatase. Detection of annexin II antibodies can also be achieved by labeling the second antibody with a fluorescent compound that includes, but is not limited to fluorescein isothiocyanate, rhodamine, phycoerythrin and Texas Red, and their detection using fluorimetry. The second antibody can also be labeled with a radioactive label or a mass label. Radioactivity can be measured using a gamma or scintillation counter depending on the nature of the radioactive substance. The measurement of antibodies reactive against annexin II can be used for the diagnosis of autoimmune or inflammatory diseases such as SLE. The monitoring of antibodies to annexin II can also be used to assess the progression of disease and efficacy of treatment. The detection of annexin II antibodies in a biological sample can be achieved by various methodologies that include, but are not limited to, immunoassays such as Western blot, ELISAs, radioimmunoassay, immunoprecipitation assays, protein A immunoassays and fluorescent immunoassays; and immuno-affinity chromatography.

To determine whether antibodies to annexin II are cross-reactive with dsDNA, antibodies to annexin II, having been eluted from the solid phase by methods well known in the art, are then interacted with native DNA that has been anchored to a separate solid phase, and eluted with a high salt buffer. Material that does not bind to the column is collected in the “flow-through” are referred to as antibodies to annexin II that do not display cross-reactivity to DNA. Immunoglobulins that bind to the DNA column are indicative of antibodies that display an affinity to both annexin II and DNA.

C. Inflammatory or Immunological Disorders to be Assessed or Treated

The disclosed compositions can be used to assess or treat one or more symptoms or tissue or organ pathology of an inflammatory or immunological disorder such as lupus nephritis or SLE, or other exemplary immunological disorders as listed above. For treatment purposes, the compositions are typically administered in an amount effective to inhibit or reduce the binding or internalization of anti-dsDNA antibodies by cells expressing annexin II on their surface.

1. Competitive Binding

In one embodiment, the compositions are administered to a subject in need thereof in an amount effective to competitively bind to annexin II expressed on the surface of cells or to anti-dsDNA antibodies and reduce or inhibit the binding between annexin II and anti-dsDNA antibodies. By limiting the amount of annexin II available for binding by anti-dsDNA antibodies, the amount of anti-dsDNA antibodies that can bind to cells or be internalized into the cells is reduced. This results in reduced induction of abnormal cell functions by anti-dsDNA antibodies, such as the synthesis and/or secretion of inflammatory mediators an example of which is IL-6, and thus reduced severity of or organ damage due to disease.

2. Soluble Annexin II or its Fragments or Molecules that Mimick Annexin II

Soluble annexin II or fragments of annexin II or mimicry molecules that are capable of binding to anti-dsDNA antibodies can be used to compete with endogenous annexin II expressed on cell surface and can therefore be an effective means for treating inflammatory responses and immune diseases or disorders. The term ‘soluble annexin II” refers to annexin II or a fragment thereof or a mimicry molecule that is not membrane-bound and is present in a subject's circulatory system. Alternatively, soluble annexin II can be administered as a bolus to an individual in need thereof to temporarily increase serum levels of soluble annexin II which can bind to anti-dsDNA antibodies.

Another method for treating an inflammatory response or autoimmune disease is by administering to an individual in need thereof a nucleic acid construct encoding soluble annexin II, or a functional fragment thereof. Functional fragment means an annexin II fragment that interferes with or inhibits or reduces anti-dsDNA antibodies internalization into cells.

In another embodiment, an annexin fusion protein can be administered to an individual in need thereof in an amount effective to reduce or inhibit anti-dsDNA antibody-mediated inflammation or a symptom thereof. The annexin II fusion proteins are discussed above. Alternatively, a nucleic acid construct encoding the annexin II fusion can be administered to an individual in need thereof wherein the nucleic acid construct is expressed in the individual and produces annexin II fusion protein in amounts effective to reduce or inhibit anti-dsDNA antibody internalization into cells.

3. Gene Delivery

Nucleic acids encoding annexin II ligands or ligands for anti-dsDNA antibodies can be administered to an individual in need thereof in an amount effective to treat an inflammatory response or immune disease. DNA delivery involves introduction of a “foreign” DNA into a cell and ultimately, into a live animal. Gene delivery can be achieved using viral vectors or non-viral vectors. One approach includes nucleic acid transfer into primary cells in culture followed by autologous transplantation of the ex vivo transformed cells into the individual, either systemically or into a particular organ or tissue.

Nucleic acid therapy can be accomplished by direct transfer of a functionally active DNA into mammalian somatic tissue or organ in vivo. DNA transfer can be achieved using a number of approaches described below. These systems can be tested for successful expression in vitro by use of a selectable marker (e.g., G418 resistance) to select transfected clones expressing the DNA, followed by detection of the presence of the expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

Retroviral-mediated human therapy utilizes amphotrophic, replication-deficient retrovirus systems (Weiss and Taylor, Cell, 82:531-533 (1995)). Such vectors have been used to introduce functional DNA into human cells or tissues, for example, the adenosine deaminase gene into lymphocytes, the NPT-II gene and the gene for tumor necrosis factor into tumor infiltrating lymphocytes. Retrovirus-mediated gene delivery generally requires target cell proliferation for gene transfer (Bordignon et al. Science 270:470-475 (1995)). This condition is met by certain of the preferred target cells into which the present DNA molecules are to be introduced, i.e., actively growing tumor cells. Gene therapy of cystic fibrosis using transfection by plasmids using any of a number of methods and by retroviral vectors has been described by U.S. Pat. No. 5,240,846 to Collins et al.

The DNA molecules encoding the annexin II or anti-dsDNA antibody ligand or fusion proteins may be packaged into retrovirus vectors using packaging cell lines that produce replication-defective retroviruses, as is well-known in the art (sees for example Stone, D. et al. J. Endocrinology, 164:103-118 (2000)). Additional viruses for gene delivery are described in Reynolds, P. N. et al, Mol Med Today, 5:25-31 (1999)).

Other virus vectors may also be used, including recombinant adenoviruses (Murphy et al. Proc Natl Acad Scii 94:13921-13926 (1997)), herpes simplex virus (HSV) for neuron-specific delivery and persistence (Lowenstein, P. R. et al., Brain Res. Mol Brain Res, 30:169-175 (1995)). Advantages of adenovirus vectors for human gene therapy include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organism. Adeno-associated virus is also useful for human therapy (Samulski, R. J. et al., EMBO J. 10:3941-3950 (1991).

Another vector which can express the disclosed DNA molecule and is useful, particularly in humans, is vaccinia virus, which can be rendered non-replicating (Peplinkski, G. R. et al., Surg Oncol Clin N Am, 7: 575-588 (1998)). Descriptions of recombinant vaccinia viruses and other viruses containing heterologous DNA and their uses in immunization and DNA therapy are reviewed in: Moss, B., Curr. Opin. Genet. Dev. 3:86-90 (1993); Moss, B. Biotechnology 20: 345-362 (1992); Moss, B., Curr Top Microbiol Immunol 158:25-38 (1992); Moss, B., Science 252:1662-1667 (1991); Piccini, A et al., Adv. Virus Res. 34:43-64 (1988); Moss, B. et al., Gene Amplif Anal 3:201-213 (1983).

In addition to naked DNA or RNA, or viral vectors, engineered bacteria may be used as vectors. A number of bacterial strains including Salmonella, BCG and Listeria monocytogenes (LM) (Hoiseth & Stocker, Nature 291, 238-239 (1981); Poirier, T P et al., J. Exp. Med. 168, 25-32 (1988); (Sadoff, J. C, et al., Science 240, 336-338 (1988); Stover, C. K., et al., Nature 351, 456-460 (1991); Aldovini, A. et al., Nature 351, 479-482 (1991); Schafer, R., et al., J. Immunol 149, 53-59 (1992); Ikonomidis, G. et al., J. Exp. Med. 180, 2209-2218 (1994)). These organisms display two promising characteristics for use as vaccine vectors: (1) enteric routes of infection, providing the possibility of oral vaccine delivery; and (2) infection of monocytes/macrophages thereby targeting antigens to professional antigen presenting cells.

In addition to virus-mediated gene transfer in vivo, physical means well-known in the art can be used for direct transfer of DNA, including administration of plasmid DNA (Wolff et ala, Science, 247:1465-1468 (1990); Hickman, M. A, et al. Hum. Gene Ther., 5:1477-1483 (1994)) and particle-bombardment mediated gene transfer (O'Brien, J. et al. Brain Res Brain Res Protco, 10:12-15 (2002)). Furthermore, electroporation, a well-known means to transfer genes into cell in vitro, can be used to transfer DNA molecules to tissues in vivo (Titomirov, A. V. et al., Biochim. Biophys. Acta 1088:131 ((1991)).

“Carrier mediated gene transfer” has also been described (Wu, C. H. et al., J. Biol. Chem. 264:16985 (1989); Wu, G. Y. et al., J. Biol. Chem. 263:14621 (1988); Soriano, P. et al., Proc. Natl. Acad. Sci. USA 80:7128 (1983); Wang, C-Y. et al., Proc. Natl. Acad. Sci. USA 84:7851 (1982); Wilson, J. M. et al., J. Biol Chem. 267:963 (1992)). Preferred carriers are targeted liposomes (Liu et al. Curr Med Chem, 10:1307-1315 (2003)) such as immunoliposomes, which can incorporate acylated mAbs into the lipid bilayer. Polycations such as asialoglycoprotein/polylysine (Wu, C. H. et al., J Biol Chem. 264: 16985-16987 (1989)) may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA for transfer.

Plasmid DNA used for transfection or microinjection may be prepared using methods well-known in the art, for example using the Qiagen procedure (Qiagen), followed by DNA purification using known methods, such as the methods exemplified herein.

4. Combination Therapy

The disclosed compositions can be administered to a subject in need thereof, alone or in combination with one or more additional therapeutic agents including, but not limited to, immunosuppressive agents, e.g., antibodies against other lymphocyte surface markers (e.g., CD40) or against cytokines, other fusion proteins, e.g., CTLA41 g, or other anti-proliferatives or immunosuppressive drugs (e.g., corticosteroid, cyclophosphamide, azathioprine, mycophenolate mofetil, calcineurin inhibitors, mTOR inhibitors, or hydroxychloroquine), or other compounds that may assist in immunosuppression.

Other suitable therapeutics include, but are not limited to, anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, salicylates, acetic acid derivatives, fenamates, propionic acid derivatives, or pyrazoles. Mixtures of these non-steroidal anti-inflammatory agents may also be employed.

Representative examples of steroidal anti-inflammatory drugs include, without limitation, corticosteroids such as hydrocortisone, prednisolone, prednisone, dexamethasone, or mixtures thereof.

### IV. Kits and Assays

Kits and assays that allow the detection and measurement of immunoglobulins that demonstrate the ability to bind to annexin II and also DNA are provided. This kit typically includes a container housing solid phase supports for annexin II protein and DNA, buffers necessary for the elution of these auto-antibodies from their supports, and subsequent measurement by means such as ELISA, the latter including a solid phase in which annexin II protein and/or DNA is bound, and to which samples are added. The detection of the antibodies will be through another antibody that is specific to a region of human immunoglobulin which may be detected by means of its conjugation with an enzyme, fluorescent dye or radioactive label.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLE 1

### Reactivity of Anti-dsDNA Antibodies to a 36 kDa Protein on the Cell Surface of Mesangial Cells Detected by Western Blot Analysis

Materials and Methods

Reagents

All chemicals and secondary antibodies were purchased from Sigma (Tin Hang Technology Limited, Hong Kong) unless otherwise stated and were of the highest purity available. Tissue culture flasks were purchased from Falcon (Becton Dickinson, Hong Kong). Culture media, supplements and Alexa Fluor 594 nm conjugated anti-mouse secondary antibody were purchased from Invitrogen Hong Kong Limited. Ninety-six well microtitre plates were purchased from Dynex Techologies (Hong Kong). Mouse anti-human annexin II antibody and goat anti-human annexin II antibody were purchased from Zymed (Invitrogen Hong Kong Limited) and Santa Cruz Biotechnology (Gene Company, Hong Kong) respectively. Goat anti-human IgG F(ab) was purchased from Biosource Int, Hong Kong. Whatman Schleicher & Schuell Protran Nitrocellulose transfer membrane was purchased from Schleicher and Schuell Bioscience GmbH Scientific. All reagents for gel electrophoresis were purchased from BioRad, Hong Kong. Enhanced chemiluminescence (ECL) reagents for Western blotting were purchased from GE Healthcare Life Science.

Culture of Human Mesangial Cells.

Primary cultures of human mesangial cells were established from nephrectomized kidneys and maintained in RPMI 1640 medium supplemented with 15% FBS. All experiments were performed on cells of the 5-7^{th} passage that had been growth-arrested for 72 h. Cells were stimulated with either normal human IgG or human IgG anti-dsDNA antibodies (final IgG concentration at 10 μg/ml) for periods up to 24 h.

Isolation of Plasma Membrane Preparation from Human Mesaongial Cells.

Confluent growth arrested human mesangial cells were washed with 15 mM HEPES buffer (Invitrogen, Hong Kong), and the plasma membrane fraction obtained according to the method of tin et al with slight modifications. The protein content of the plasma membrane preparations was measured using a modified Lowry assay and subsequently used for the identification of cell surface antigens through which anti-dsDNA antibodies bound.

Matrix-Assisted-Laser-Desorption-lonization Time-of-Flight (ALDI-TOF) Mass Spectrometry

Proteins immunoprecipitated from plasma membrane preparations by anti-dsDNA antibodies were electrophoresed on 10% polyacrylamide gels, stained with 0.5% Coomassie blue G-250 in 50% methanol, 10% acetic acid for 30 min and destained in 40% methanol/10% acetic acid until bands were observed. Detectable bands were excised and submitted to The Rockefeller University Proteomics Resource Centre for in-gel tryptic digestion of proteins and subsequent mass spectrometric analysis as previously described. Fernandez, J., et al. Electrophoresis 19: 1036-1045 (1998).

SDS-PAGE and Immunoblotting

Plasma membrane preparations (10 μg) were separated by SDS-PAGE and proteins were transferred onto nitrocellulose membranes using a mini-gel transfer system at 100 V for 1 h at 4° C. Equal loading of proteins was confirmed by staining of membranes with Ponceau S solution. Membranes were probed with a monoclonal antibody to human annexin II, then incubated with horseradish peroxidase-conjugated anti mouse IgG, and proteins visualized by ECL.

Results

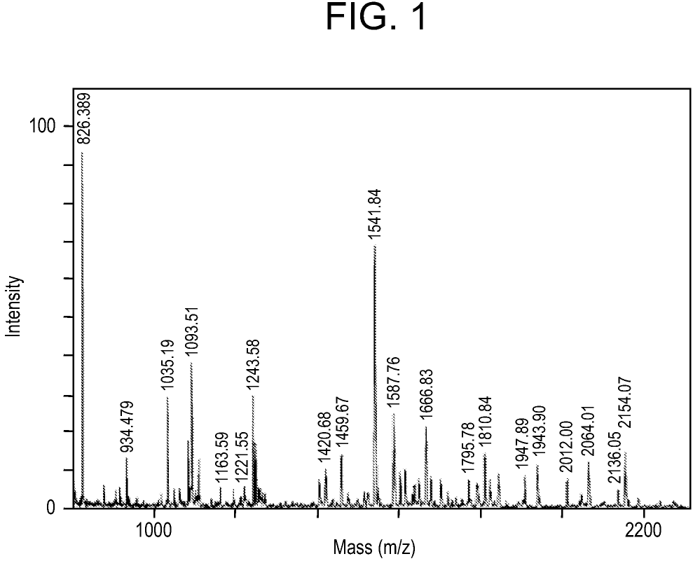

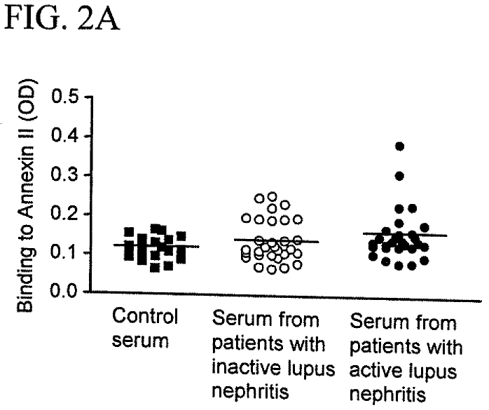

Cell surface proteins were isolated from human mesangial cells and subjected to SDS PAGE, transferred to nitrocellulose membrane and probed with a panel of anti-dsDNA antibodies. Western blot analysis revealed prominent reactivity to a band with a molecular weight of 36 kDa. This band was not reactive to control human IgG from healthy subjects. To determine the identity of this protein, additional gels were prepared and proteins visualized by Coomassie Blue. The band at 36 kDa was excised from the gel, eluted and subjected to trypsin digestion. Peptide fragments were analyzed by MALDI-TOF mass spectrometry and identified as annexin II as shown in FIG. 1.

### EXAMPLE 2

### Reactivity of Immunoglobulins in Serum Samples or Anti-dsDNA Antibody Preparations Isolated from Serum Samples of Patients with SLE to Annexin II.

Materials and Methods

Clinical Samples

Serum samples were obtained from fifty seven subjects with a diagnosis of SLE that fulfilled the American Rheumatism Association. These patients were all diagnosed with biopsy-proven diffuse proliferative lupus nephritis. These subjects comprised 52 women and 5 men (mean age 48.8±10.5 and 47.1±6.8 years respectively). All were of Chinese origin. Disease activity was assessed using the SLE disease activity index (SLEDAI). Nephritic flare (active disease) was defined by clinical manifestations together with a SLEDAI≧10 and renal biopsy showing active diffuse proliferative lupus nephritis, and remission was confirmed by a SLEDAI≦4 and quiescent urinary sediment.

An aliquot of serum sample was used to detect the presence of antibodies to annexin II protein, whilst another aliquot was used to isolate anti-dsDNA antibodies.

Isolation of Human Polyclonal Anti-dsDNA Antibodies

Polyclonal IgG anti-double stranded (ds) DNA antibodies were isolated from sera of patients with lupus nephritis using sequential affinity chromatography, and the IgG level determined as previously described. Chan, T. M., et al. J Am Soc Nephrol. 13: 1219-1229 (2002).

Measurement of Human Anti-dsDNA Antibody Titers

Anti-dsDNA activity was determined in human serum samples and anti-dsDNA antibody preparations using a commercial ELISA according to the manufacturer's instructions (Microplate autoimmune anti-dsDNA quantitative ELISA, BioRad, Hong Kong).

Assessment of Immunoglobulin Binding to Annexin II by ELISA

Ninety six well microtitre plates were coated overnight with human recombinant annexin II (1 ng/ml) in 0.1 M carbonate buffer, pH 9.6. Plates were washed thrice with PBS containing 0.05% Tween-20 between incubations and all incubations were for 1 h at 37° C. Plates were blocked with 3% BSA, and incubated with serum samples from healthy individuals or patients with lupus nephritis (starting dilution 1:100), control IgG, or isolated human polyclonal anti-dsDNA antibody preparations (10 μg/ml, final IgG concentration) added in triplicate in serial dilution. Plates were then incubated with horseradish peroxidase-conjugated goat anti-human IgG (5 μg/ml). Bound IgG was detected by the addition of o-phenylenediamine for 30 min, and the absorbance measured at 450 nm. An OD greater than the mean of control sera or IgG +2SD was considered significant.

Results

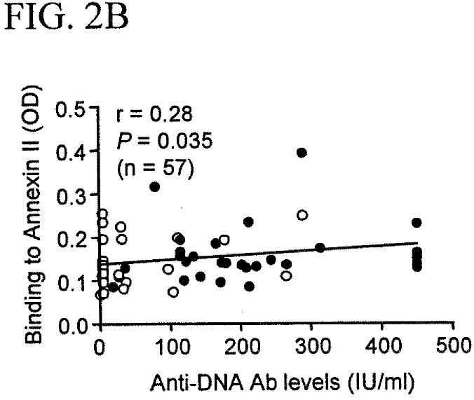

The prevalence of annexin II-binding activity in the sera of patients with lupus nephritis was investigated. Sixteen of the 57 serum samples obtained from lupus patients exhibited significant immunoglobulin reactivity to annexin II. This reactivity of serum samples to annexin II correlated with the patients' circulating anti-dsDNA antibody levels as shown in FIGS. 2A and 2B.

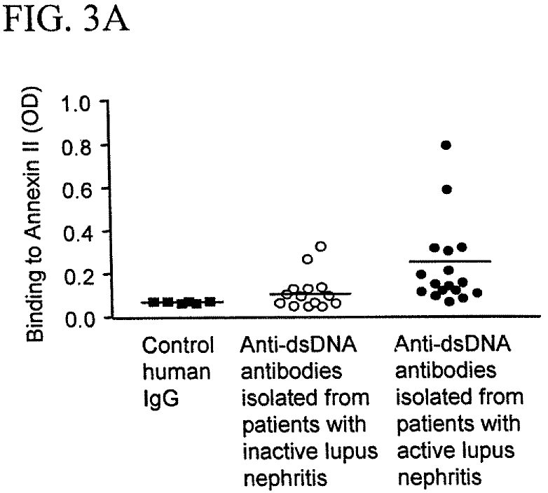

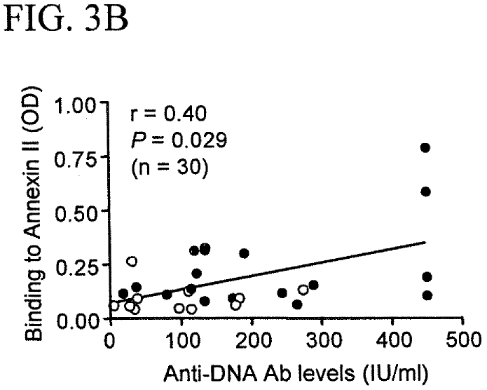

In a further study, 31 different anti-dsDNA antibodies were assessed for their annexin II-binding activity. The anti-dsDNA antibodies were separated into those that were isolated from patients during remission (n=14) and those from patients with active disease (n=17). 8 of the 14 antibody preparations obtained during remission and 16 of the 17 antibodies obtained during active disease demonstrated significant reactivity for annexin II (FIGS. 3A-3B.). Annexin II reactivity by anti-dsDNA antibodies correlated with circulating anti-dsDNA antibody titres. These results thus confirm the clinical relevance of the binding between anti-dsDNA antibodies and annexin II, and their association with disease activity.

### EXAMPLE 3

### Expression of Annexin II in the Kidney in Disease or Healthy State, and Its Co-Localization with Immunoglobulin Deposition.

Materials and Methods

Animal Studies

Female NZBWF1/J mice were purchased from The Jackson Laboratory (Bar Harbor, USA). All procedures were carried out according to institutional guidelines and regulations for animal care. Ten mice were followed weekly from the age of 15 weeks to assess proteinuria and anti-dsDNA antibody titres using dip-stick and ELISA respectively. Once fixed proteinuria >300 mg/dl was noted on two separate occasions two days or more apart (normally 20 weeks of age), mice were sacrificed 2, 6 and 12 weeks (n=4, 3 and 3 respectively) after onset of proteinuria, and kidneys harvested for histological studies and assessment of annexin II expression.

In a separate study, female BALB/c and pre-diseased NZBWF1/J mice (15 weeks of age, n=10 for both groups) were given 100 μg human polyclonal anti-dsDNA antibodies or control human IgG in 100 μl saline by intraperitoneal injection for 10 consecutive days. Mice were sacrificed, kidneys harvested, and snap-frozen for immunohistochemical analysis of annexin II expression and deposition of human anti-dsDNA antibodies.

Cytochemical and Immunohistochemical Staining of Renal Specimens

15 renal biopsies showing active diffuse proliferative lupus nephritis were studied, Normal renal tissue from 5 patients who underwent nephrectomy for tumor was included as control. Paraffin sections of NZBWF1/J mouse kidneys obtained during different stages of disease were also studied. Samples were washed thrice with PBS in between all steps and unless otherwise stated, all incubations were for 1 h at 37° C. To detect annexin II expression, paraffin sections were incubated with mouse anti-annexin 11 antibody overnight at room temperature. Samples were then incubated with horseradish peroxidase-conjugated anti-mouse antibody. Signal detection was by the peroxidase-anti-peroxidase method (Dako, Gene Company, Hong Kong) with hematoxylin counterstaining.

To study co-localization of annexin II and IgG, renal cryosections (5 μm) from patients with lupus nephritis were incubated with mouse anti-human annexin II and then with Alexa Fluor 594 nm conjugated anti-mouse secondary antibody and FITC-conjugated goat anti-human IgO. To assess annexin II and C3 co-localization, FITC-conjugated anti-human C3 was used. After washing with PBS, sections were mounted with fluorescence mountant and epifluorescence viewed using a BioRad Radiance 2100 confocal microscope or Axiovert 135 microscope. Samples from patients with primary non-lupus glomerular diseases were used as controls.

To assess the deposition of human anti-dsDNA antibodies in murine renal sections obtained from BALB/c and NZBWF1/J mice, samples were incubated with goat anti-human IgG. Annexin II expression was detected using rabbit polyclonal antibody to annexin II.

Results

Annexin II expression in kidney biopsies from patients with active lupus nephritis was examined, in particular its localization in relation to immunoglobulin deposition, and compared to normal kidney tissue and biopsy specimens from patients with non-lupus renal parenchymal diseases. Cytochemical staining of biopsy specimens from patients with lupus nephritis showed intense renal expression of annexin II compared with normal renal tissue. Glomerular expression of annexin II was up-regulated in patients with lupus nephritis, especially in the mesangium. Increased annexin II expression in the kidneys of patients with lupus nephritis co-localized with IgG and C3 depositions as demonstrated by immunohistochemical staining. Kidney biopsies from patients with non-lupus renal parenchymal diseases (such as IgA nephropathy, diabetic nephropathy and membranoproliferative glomerulonephritis) showed only constitutive but no significant increase in annexin II expression, and there was no co-localization with IgG deposition. In summary, increased annexin II expression and its co-localization with IgG and C3 deposition occurred only in kidney samples from patients with lupus nephritis.

Since it is not feasible to perform frequent longitudinal renal sampling in patients, the NZBWF1/J mouse was used to investigate the expression of annexin II over time. The NZBWF1/J mouse is an established animal model of lupus nephritis, in which the mice exhibit similar features of disease manifestations as humans. Renal sections from mice were obtained at different stages of disease and stained for annexin II expression. Progressive renal injury in NZBWF1/J mice was accompanied by increasing renal expression of annexin II, and it is expressed most intensely in mice with established disease.

To determine whether anti-dsDNA antibodies contributed to the IgG staining that was shown to co-localize with annexin II in kidney biopsies from patients with lupus nephritis, pre-diseased NZBWF1/J mice or BALB/c mice (non-autoimmune mouse strain which acted as a control) were administered either human anti-dsDNA antibodies or control human IgG by intra-peritoneal injection on 10 consecutive days. The kidneys were dissected, and cytochemical analysis performed. Anti-dsDNA antibodies but not control human IgG was shown to deposit in the kidney of only the NZBWF1/J mice, and which co-localized with annexin II.

### EXAMPLE 4

### In vitro Studies to Investigate the Functional Consequences of Anti-dsDNA Antibody Binding to Annexin II in Mesangial Cells

Measurement of IL-6 concentration in culture supernatant

Human mesangial cells were incubated with anti-dsDNA antibodies or control IgO for 24 h. The supernatant was decanted, centrifuged at 2000 g for 10 min to remove cell debris, and the level of IL-6 measured using a commercial ELISA kit according to the manufacturer's instructions (Pharmingen, Bio-Gene, Hong Kong). The results were expressed as ng/μg cellular protein.

Annexin II Knockdown Using Stealth RNAi

Human mesangial cells were cultured until 50% confluent and transfected with duplex Stealth RNAi specific for annexin II (Stealth Select HSS141224, Invitrogen, Hong Kong) or Stealth RNAi negative control (Stealth RNAi Negative Control Med GC, Invitrogen, Hong Kong) complexed with Lipofectamine 2000 at a final concentration of 40 nM Stealth RNAi. Transfection efficiency was monitored by BLOCK-iT™ Fluorescent Oligo according to the manufacturer's instructions. After 4 h incubation, the RNAi-Lipofectamine complex was removed and cells cultured overnight in RPMI 1640 medium supplemented with 15% FBS. Cells were subsequently growth arrested for 72 h, prior to incubation with anti-dsDNA antibodies (final IgG concentration 10 μg/ml), control IgG, or serum free medium.

Results

To ascertain the functional consequence of the interaction between anti-dsDNA antibody and annexin II in mesangial cells, human mesangial cells were incubated with either control human IgG or anti-dsDNA antibodies for 24 h, after which time, the culture media was decanted and its level of IL-6 was measured. IL-6 was chosen because it is an established mediator of inflammation and mesangial cell proliferation, and it has the ability to induce B- and T-cell activation and differentiation which result in increased production of anti-dsDNA antibodies. Additionally, IL-6 has the ability to exacerbate glomerulonephritis in lupus patients. Lupus flare is associated with increased serum IL-6 levels.

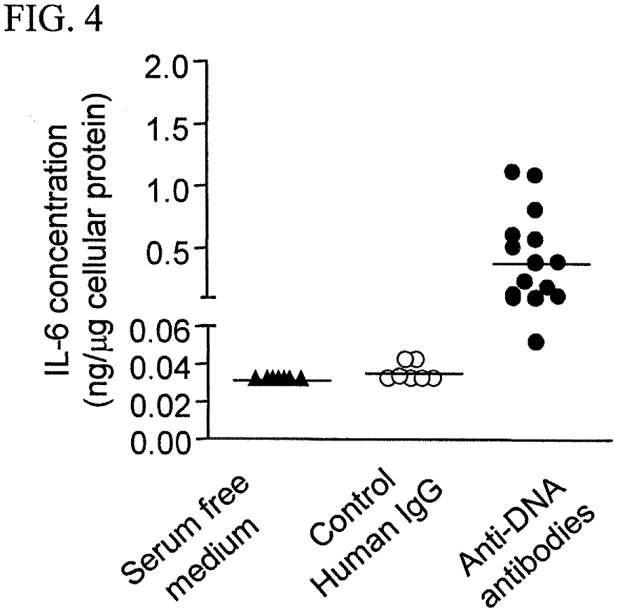

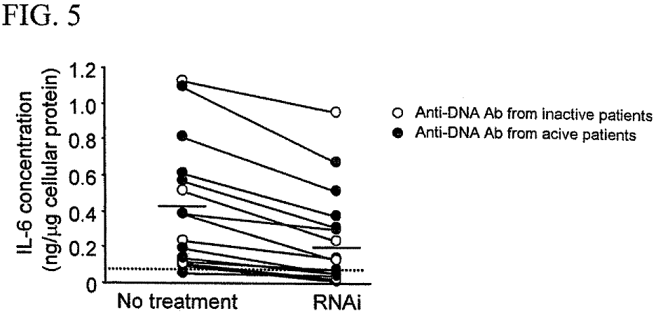

Binding of anti-dsDNA antibodies to human mesangial cells resulted in a significant increase in IL-6 secretion compared to cells incubated with culture medium alone, or control human IgG (FIG. 4). Gene silencing of annexin II protein using RNAi inhibited anti-dsDNA binding, their internalization and translocation to the cell nuclei. Knockdown of annexin II protein expression was also accompanied by a decrease in IL-6 secretion (FIG. 5).

In summary, patients with lupus nephritis have increased renal expression of annexin II. Data highlights the interaction of annexin II with anti-dsDNA and the interaction of the latter with the complement system during disease manifestations which contribute to the inflammatory processes of this disorder. Following the binding of anti-dsDNA antibodies to annexin II, antibodies are internalized and translocated to the cell cytoplasm and/or nucleus whereby they elicit alterations in cellular functions and the induction of disease mediators such as but not limited to IL-6.

Statistical Analyses

All experiments were repeated three times. Results are expressed as mean±SD. Statistical analysis was performed using GraphPad Prism version 3.00 for Windows®, (GraphPad Software, San Diego, Calif., USA). Differences were assessed by paired two-tailed Student t-test or one-way ANOVA. Correlation analyses were examined using the Spearman's method. Two-tailed P<0.05 was considered statistically significant.

1. (canceled)

2. (canceled)

3. (canceled)

4. A composition for inhibiting or reducing binding of anti-DNA antibodies to cell surface annexin II and subsequent cellular internalization of anti-DNA antibodies comprising an effective amount of an inhibitory nucleic acid to down regulate cellular expression of annexin II and a sterile pharmaceutically acceptable carrier for administration to an individual in need thereof.

5. The composition of claim 4 wherein the inhibitory nucleic acid is selected from the group consisting of dsRNA, siRNA, microRNA, antisense DNA or combinations thereof.

6. The composition of claim 5, wherein the dsRNA is at least about 21 nucleotides.

7. A method for treating an inflammatory disorder or an immunological disorder comprising
administering an effective amount of the composition of any of claims 4 to 6 to a subject in need thereof.

8. (canceled)

9. (canceled)

10. (canceled)

11. The composition of claim 4 wherein the inhibitory nucleic acid is in an affective amount to treat an inflammatory or immunological diorder selected from the group consisting of systemic lupus erythematosus, lupus nephritis, IL-6 mediated inflammation, rheumatoid arthritis, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

12. (canceled)

13. (canceled)

14. The method of claim 7 wherein the inflammatory or immunological disorder is an autoimmune disease.

15. The method of claim 14 wherein the inflammatory or immunological disorder is systemic lupus erythematosus.

16. (canceled)

17. The method of claim 7 wherein the inflammatory or immunological disorder is selected from the group consisting of rheumatoid arthritis, systemic lupus erythematosus, lupus nephritis, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.
